(19) United States
(12) Patent Application Publication
(10) Pub. No.: US 2022/0125282 A1
(43) Pub. Date: Apr. 28, 2022

(54) SYSTEMS, DEVICES AND METHODS FOR ADVANCING MEDICAL INSTRUMENTS THROUGH A WORKING CHANNEL

(71) Applicant: Prosper ABITBOL, Boca Raton, FL (US)

(72) Inventor: Prosper ABITBOL, Boca Raton, FL (US)

(21) Appl. No.: 17/513,456

(22) Filed: Oct. 28, 2021

Related U.S. Application Data
(60) Provisional application No. 63/106,737, filed on Oct. 28, 2020.

(51) Int. Cl. A61B 1/00; A61B 1/018

(52) U.S. Cl. CPC A61B 1/00133; A61M 25/0113; A61B 1/018

(57) ABSTRACT

Systems, devices and methods may include a device for feeding an endoscopy operating instrument into an endoscope. The device may include a housing coupled to a proximal end of the endoscope. The device may include a mechanism to advance the medical instrument in a rotational manner through the housing. The device may include a member forming an internal working channel and having an entry port, exit port, feeding system, and coupling structure.

### CROSS REFERENCE TO RELATED APPLICATIONS

This application claims priority to U.S. Provisional Application No. 63/106,737 filed Oct. 28, 2020 the entire contents which are hereby incorporated by reference.

### BACKGROUND

### 1. Technical Field

The invention relates to systems, devices and methods for advancing medical instrument(s) through a working channel and, more particularly, feeding endoscopy instrument(s) through a working channel of an endoscope in combination with providing for rotation of the medical instrument. The field of the currently claimed embodiments of this invention relates to systems and devices for feeding an endoscopy operating instrument into an endoscope.

### 2. Discussion of Related Art

An endoscopy may be a procedure in which a body is examined internally using a medical device called an endoscope. An insertion tube of the endoscope may be a long, flexible tube which is inserted into a patient during the endoscopy. During the endoscopy, an endoscopy operating instrument is manually passed through the insertion tube of the endoscope. Insertion of the endoscopy operating instrument is cumbersome, labor-intensive, and error prone. Positioning, removal and cleaning of the endoscopy operating instrument can be challenging.

Thus, there is a need for a device, system and method to facilitate insertion and use of an endoscopy operating instrument during an endoscopy.

### SUMMARY

An embodiment of the invention relates to a device for advancing a medical instrument through an endoscope, including: a housing configured to be coupled to a proximal end of the endoscope; and a mechanism in the housing configured to advance the medical instrument in a rotational manner through the housing and into the endoscope.

An embodiment of the invention relates to the device above, where the housing forms a working channel passing through the housing, and wherein the working channel is configured to extend between an inlet and an outlet.

An embodiment of the invention relates to the device above, where the mechanism in the housing is configured to advance the medical instrument in a rotational manner includes a pair of rotatable pins coupled to the housing and configured to propel the medical instrument through the working channel.

An embodiment of the invention relates to the device above, where the pair of rotatable pins are offset.

An embodiment of the invention relates to the device above, where the working channel includes a helical shape.

An embodiment of the invention relates to a device for feeding an endoscopy operating instrument into an endoscope, including: a member forming and internal working channel, the internal working channel configured to surround a portion of the endoscopy operating instrument, and the member further including: an input structure positioned at an anterior end of the member; an entry port formed by the input structure, the entry port providing access to a first end of the internal working channel; an exit port disposed on a posterior end of the member, the exit port providing access to a second end of the internal working channel; a feeding system mechanically coupled to the member, the feeding system configured to propel the endoscopy operating instrument through the internal working channel; and a coupling structure positioned at a posterior end of the member, the coupling structure configured to mechanically couple the member to the endoscope. In such an embodiment, the internal working channel includes a helical shape, and the feeding system is further configured to propel the endoscopy operating instrument in a first direction from the anterior end of the member to the posterior end of the member, and alternately in a second direction from the posterior end of the member to the anterior end of the member.

An embodiment of the invention relates to the device above, where the internal working channel is configured to rotate the endoscopy operating instrument between 1 and 10 rotation(s) per 100 centimeters as the feeding system propels the endoscopy operating instrument through the internal working channel. In some embodiments, the internal working channel is configured to rotate the endoscopy operating instrument between 1 and 5 rotation(s) per 100 centimeters as the feeding system propels the endoscopy operating instrument through the internal working channel.

An embodiment of the invention relates to the device above, where the internal working channel includes a diameter that is greater than the diameter of the working channel of the endoscope. In some embodiments, the internal working channel of the device is between 60% to 80% greater than the diameter of the working channel of the endoscope.

An embodiment of the invention relates to the device above, where the feeding system is configured to be in communication with a controller.

An embodiment of the invention relates to the device above, where the feeding system is a pair of parallel pins.

An embodiment of the invention relates to the device above, where the parallel pins are offset from each other.

An embodiment of the invention relates to the device above, where the parallel pins include a drive wheel, and an idle wheel.

An embodiment of the invention relates to the device above, where the parallel pins are configured such that the endoscopy operating instrument passes between the parallel pins when the device is in use.

An embodiment of the invention relates to the device above, where at least one of the parallel pins is configured such that the at least one of the parallel pins can be adjusted to be a predetermined distance from a second of the parallel pins.

An embodiment of the invention relates to the device above, where the input structure includes a funnel shape.

An embodiment of the invention relates to a system for feeding an endoscopy operating instrument into an endoscope, including: a controller; and a device for feeding the endoscopy operating instrument, the device including: a member forming and internal working channel, the internal working channel configured to surround a portion of the endoscopy operating instrument, and the member further including: an input structure positioned at an anterior end of the member; an entry port formed by the input structure, the entry port providing access to a first end of the internal working channel; an exit port disposed on a posterior end of the member, the exit port providing access to a second end of the internal working channel; a feeding system mechanically coupled to the member, the feeding system configured to propel the endoscopy operating instrument through the internal working channel; and a coupling structure positioned at a posterior end of the member, the coupling structure configured to mechanically couple the member to the endoscope. In such an embodiment, the internal working channel includes a helical shape, the feeding system is configured to be in communication with a controller, and the feeding system is further configured to propel the endoscopy operating instrument in a first direction from the anterior end of the member to the posterior end of the member, and alternately in a second direction from the posterior end of the member to the anterior end of the member.

An embodiment of the invention relates to the system above, where the controller is configured instruct the feeding system to propel the endoscopy operating instrument for at least one of a predetermined length of time, a predetermined distance, and a predetermined speed.

An embodiment of the invention relates to the system above, where the internal working channel is configured to rotate the endoscopy operating instrument between 1 and 10 rotation(s) per 100 centimeters as the feeding system propels the endoscopy operating instrument through the internal working channel. In some embodiments, the internal working channel is configured to rotate the endoscopy operating instrument between 1 and 5 rotation(s) per 100 centimeters as the feeding system propels the endoscopy operating instrument through the internal working channel.

An embodiment of the invention relates to the device above, where the internal working channel includes a diameter that is greater than the diameter of the working channel of the endoscope. In some embodiments, the internal working channel of the device is between 60% to 80% greater than the diameter of the working channel of the endoscope.

An embodiment of the invention relates to the system above, where the feeding system is a pair of parallel pins.

An embodiment of the invention relates to the system above, where the parallel pins are offset from each other.

An embodiment of the invention relates to the system above, where the parallel pins include a drive wheel, and an idle wheel.

An embodiment of the invention relates to the system above, where the parallel pins are configured such that the endoscopy operating instrument passes between the parallel pins when the device is in use.

An embodiment of the invention relates to the system above, where at least one of the parallel pins is configured such that the at least one of the parallel pins can be adjusted to be a predetermined distance from a second of the parallel pins.

An embodiment of the invention relates to the system above, where the input structure includes a funnel shape.

An embodiment of the invention relates to a method of feeding an endoscopy operating instrument into an endoscope, including coupling the device above to the endoscope; feeding the endoscopy operating instrument into the device above; and actuating the device above such that the device advances the endoscopy operating instrument into the endoscope in a rotational manner.

An embodiment of the invention relates to a method of cleaning an endoscope, including coupling the device above to an endoscope; feeding a cleaning instrument into the device above; actuating the device above such that the device advances the cleaning instrument into the endoscope in a rotational manner; and actuating the device such that the device pulls the cleaning instrument from the endoscope.

### BRIEF DESCRIPTION OF THE DRAWINGS

Further objectives and advantages will become apparent from a consideration of the description, drawings, and examples.

FIG. 1 is a perspective side view of a device for feeding an endoscopy operating instrument into an endoscope according to an embodiment of the invention.

FIG. 2 is an exploded view of the device of FIG. 1.

FIG. 3 is a perspective bottom view of the device of FIG. 1.

FIG. 4 is a perspective top view of the device of FIG. 1

FIG. 5 is a perspective front view of the device of FIG. 1.

FIG. 6 is a perspective side transparent view of the device of FIG. 1.

FIG. 7 is a system incorporating the device for feeding an endoscopy operating instrument into an endoscope according to an embodiment of the invention.

FIG. 8 is a flow chart depicting a method of use of the device and system described above for advancing an endoscopy operating instrument into an endoscope during an endoscopy according to an embodiment of the invention.

FIG. 9 is a flow chart depicting a method of use of the device and system described above for cleaning an endoscope according to an embodiment of the invention.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

This disclosure is generally directed to an endoscopy procedure in which a body is examined internally using an endoscope. An insertion tube of the endoscope may be a long, flexible tube which is inserted into a patient during the endoscopy. During the endoscopy, endoscopy operating instrument(s) may be passed manually through the insertion tube of the endoscope. The placement and orientation of the endoscopy operating instrument with manual insertion may be challenging and causes the surgeon either rotate the entire endoscope to reorient the endoscopy operating instrument or pull the entire endoscope out of the patient and reinsert it such that the endoscopy operating instrument is reintroduced into the patient in the correct orientation. The devices, systems and methods described herein provide for a feeding and/or advancing mechanism for insertion into the endoscope and may further provide for the orientation and/or rotation of the endoscope operating instrument while positioned in the endoscope in the patient.

Following a procedure, when the endoscope has been removed from the patient, the endoscope insertion tube may be manually cleaned by a technician, which is labor-intensive and inefficient. During the cleaning procedure, the technician manually feeds a cleaning cable having a cleaning brush into the insertion tube and scrubs the interior of the insertion tube by moving the cleaning cable into and out from the insertion tube, which may be ineffective, inefficient and labor intensive. The devices, systems and method described herein provide for a feeding and/or advancing mechanism that may be used for this cleaning procedure.

As describe herein, a device, system and method is provided that facilitates insertion and/or orientation of an endoscopy operating instrument during an endoscopy, and that may be used for reliable cleaning of an endoscope following a procedure.

As used throughout, an “endoscope” refers to a tubular instrument which can be introduced into a subject to give a view of the subject's internal parts during an endoscopy. An “endoscopy” is a procedure where the inside of a body is examined using the endoscope. The endoscope may include a flexible insertion tube that may have a light source and/or a camera at one end. Images of the inside of the body are relayed to a display screen. The flexible insertion tube may be guided through (into) a body cavity via mechanisms controlled externally by a user. The flexible insertion tube can be inserted into the body through a natural opening, such as into the mouth and down the throat, or through the anal cavity. The flexible insertion tube can also be inserted through a small cut or incision made in the skin when keyhole surgery is being carried out. The endoscope contains an internal working channel that provides access to the flexible insertion tube. During an endoscopy, a surgical tool, or endoscopy operating instrument, is inserted into the working channel and through the flexible insertion tube.

The endoscopy can be used to investigate unusual symptoms, including but not limited to, difficulty swallowing (dysphagia), persistent abdominal pain, chest pain that is not caused by heart-related conditions, persistent nausea and vomiting, unexplained weight loss, vomiting blood, persistent diarrhea, and blood in stool. The endoscope can also help perform certain types of surgery, and/or remove a small sample of tissue for further analysis (e.g., a biopsy).

Examples of endoscopes are known to one of ordinary skill in the art, and are described, for example in U.S. Patent Application Publication 2018/0160885, published on Jun. 14, 2018, and which represents the instant inventor's own work. The entire contents of U.S. Patent Application Publication 2018/0160885 are hereby incorporated by reference. Specific examples may include, gastroscopes, colonoscopes, endoscopic retrograde cholangiopancreatography scopes, sigmoidoscopes, push-enteroscopes, and double-balloon endoscopes.

As used throughout, an “endoscopy operating instrument” is a medical device that is passed through an endoscope during an endoscopy and is used to perform a surgical procedure. Examples of such instruments are known to one of ordinary skill in the art, and include, biopsy forceps (cold and hot), snares (cold and hot), heated probes, argon plasma coagulation probes, needle injectors, clips, sphincterotome probes, needle knife probes, baskets and balloons.

Embodiments of the instant invention provide a disposable device that advances and rotates an endoscopy instrument through the working channel of the of an endoscope. Such a device eliminates the repetitive motions of feeding the endoscopy instruments manually, thereby reducing the duration of the procedures and reducing fatigue and injuries to an operator. In some such embodiments, the device is operated by a foot pedal. The device will advance the endoscopy instrument in pre-programed distance depending on the type of endoscope use. The rotating function of the device uniquely offers the operator an additional option of targeting endoscopic lesions. By rotating the endoscopy instrument through the device, the operator is be able to align the endoscopy instrument more precisely toward the targeted lesion.

Embodiments of the invention provide a disposable device for a method of rapidly and efficiently cleaning an endoscope. In such an embodiment, the device is operated by a foot pedal. A cleaning technician actuates the device by stepping on the foot pedal. The device will then propel and rotate a cleaning brush through the working channel of the endoscope, ensuring a higher level of cleanliness.

Embodiments of the instant invention provide a novel and non-obvious solution to the above-described problem. Specifically, embodiments of the invention relate to devices, systems, and methods for mechanically feeding the endoscopy operating instrument into the endoscope.

The instantly described devices, systems, and methods feed and/or advance a medical device and/or instrument using a rotational propulsion of the endoscopy operating instrument through the insertion tube of the endoscope, and to the target tissue. If the endoscopy operating instrument arrives at the target tissue in the incorrect orientation, a surgeon can simply, for example, pull the endoscope back and actuate the device to rotate the endoscopy operating instrument until it is oriented in the desired orientation. This allows the surgeon to reorient the endoscopy operating instrument without having to physically rotate the entire endoscope, or without having to remove the endoscope entirely and subsequently reintroduce it into the patient. As will be discussed in further detail below, the instantly described devices have a feeding system and a helical-shaped internal working channel which are configured to work in combination to advance the endoscopy operating instrument through the insertion tube of the endoscope and to the target tissue in a rotational manner.

Embodiments of the instant invention also provide a novel and non-obvious solution to the above-discussed problems associated with cleaning an endoscope following a procedure. Specifically, embodiments of the invention relate to devices, systems, and methods for mechanically feeding the endoscopy operating instrument into the endoscope. For example, a system according to an embodiment of the invention allows for a user to manipulate the rate at which the endoscopy operating instrument is fed into the insertion tube of the endoscope, and also allows for the direction of the propulsion of the endoscopy operating instrument to be manipulated (e.g. towards a distal end of the insertion tube, or towards a proximal end of the insertion tube).

The ability to manipulate the direction and rate of feeding into the insertion tube of the endoscope, combined with the rotational nature of the propulsion of the endoscopy operating instrument provide an unexpected solution to the problem of systematically and efficiently cleaning the insertion tube following use. To clean the insertion tube, a technician replaces the endoscopy operating instrument with a cleaning tool and uses the instantly disclosed device to feed the cleaning tool into the insertion tube. The technician then cleans the inside of the insertion tube by actuating the device to feed the cleaning tool into and out from the insertion tube. The rotation of the cleaning tool as it is fed into and out from the insertion tube results in cleaning of the insertion tube as the cleaning tool scrubs the walls of the working channel of the insertion tube. In addition, the technician can vary the rate at which the cleaning tool is fed into and out from the insertion tube, thus allowing for more, or less vigorous cleaning as desired. The method described above allows for systematic and efficient cleaning of the insertion tube.

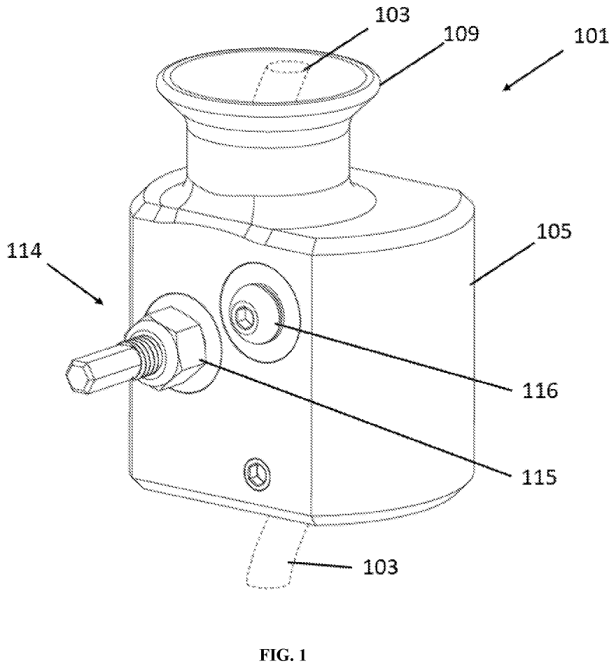

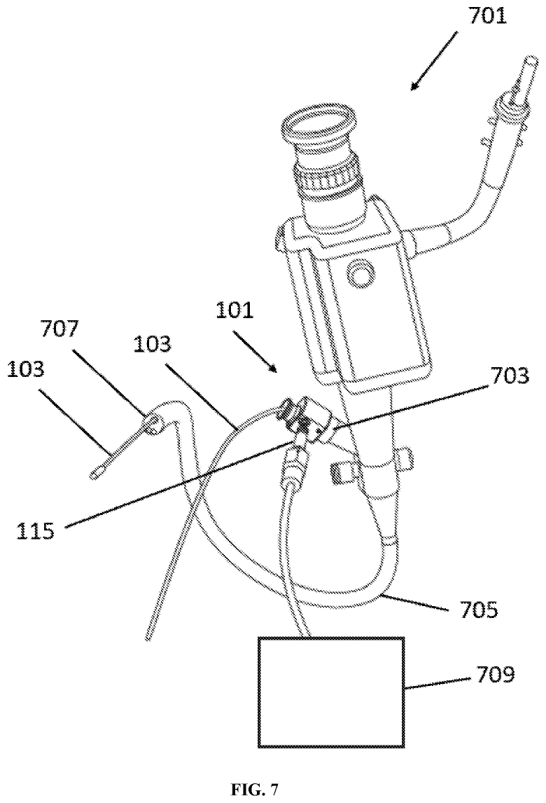

Referring to the figures, an embodiment of a device 101 is shown in accordance with the principles of the invention. FIG. 1-6 show device 101 alone and FIG. 7 shows device 101 incorporated into an endoscope. Referring to FIG. 1, device 101 is configured for feeding an endoscopy operating instrument 103 into an endoscope according to an embodiment of the invention. Device 101 generally includes an internal working channel 107 through which an instrument is passed. Internal working channel 107 is a uniquely configured pathway as described herein allowing the instrument to be feed and/or advanced through device 101 and into the endoscope in accordance with the principles of the invention. Device 101 as shown may include a member 105 forming internal working channel 107. Internal working channel 107 may be configured to surround a portion of an endoscopy operating instrument 103. Member 105 may further include an input structure 109 positioned at an anterior end of the member 105 and an entry port 111 formed by the input structure 109. Entry port 111 may provide access to a first end of the internal working channel 107. An exit port 113 may be disposed on a posterior end of member 103. Exit port 113 may provide access to a second end of the internal working channel 107. A feeding system, shown generally at 114, may be mechanically coupled to member 105. The feeding system 114 may be a pair of pins 115, 116. As shown pins 115, 116 are positioned parallel to each other. Feeding system 114 may be configured to propel endoscopy operating instrument 103 through the internal working channel 107 when in use. For example, the pair of pins 115, 116 are configured such that the endoscopy operating instrument is disposed between them when it is being advanced. At least one of the pins 115 is mechanically actuated to rotate. In some embodiments, the pin is mechanically actuated by an electrical motor (not shown). Upon actuation of the pin 115, the endoscopy operating instrument 103 is advanced through the internal working channel 107 of the device 101. The internal working channel 107 includes a helical shape, and the feeding system 114 is further configured to propel the endoscopy operating instrument 103 in a first direction from the anterior end of the member to the posterior end of the member, and alternately in a second direction from the posterior end of the member to the anterior end of the member. For example, the pair of pins 115, 116 are configured such that the endoscopy operating instrument 103 is disposed between them when it is being advanced. At least one of the pins 115 is mechanically actuated to rotate. For advancing the endoscopy operating instrument 103 in a first direction towards the posterior end of the device 101, pin 115 is mechanically actuated to rotate clockwise. This clockwise rotation causes the endoscopy operating instrument 103 to advance towards the posterior end of the device 101 and causes pin 116 to rotate counterclockwise. For advancing the endoscopy operating instrument 103 in a second direction towards the anterior end of the device 101, pin 115 is mechanically actuated to rotate counterclockwise. This counterclockwise rotation causes the endoscopy operating instrument 103 to advance towards the anterior end of the device 101 and causes pin 116 to rotate clockwise.

The device 101 of FIG. 1 also may include a coupling structure 117 positioned at a posterior end of the member 105. Coupling structure 117 may be configured to mechanically couple member 105 to the endoscope (as shown in FIG. 7). As shown, input structure 109 includes a funnel shape to facilitate insertion of the endoscopy operating instrument into internal working channel 107.

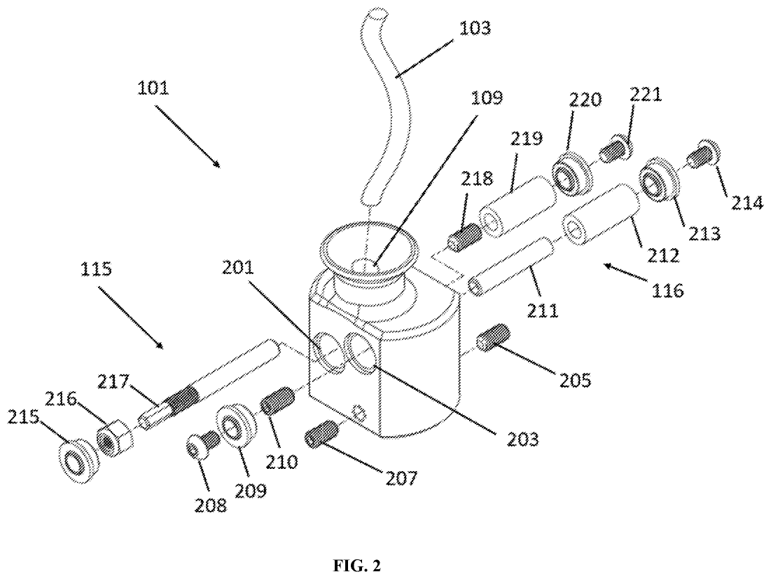

Referring to FIG. 2, an exploded view of device 101 is shown and endoscopy operating instrument 103 is shown outside of the internal working channel 107. In this embodiment, each of the pins 115, 116 comprise a series of screws 208, 210, 214, 215, 218, 221, bolts, 209, 213, 216, 220, pins 211, 217, and rollers 212, 219. Member 105 may be adapted to receive the individual parallel pins through openings 201, 203. FIG. 2 also shows components of the coupling structure 117. Specifically, coupling structure 117 as shown includes two (2) opposing screws 205, 207 which can be tightened to secure device 101 to an endoscope.

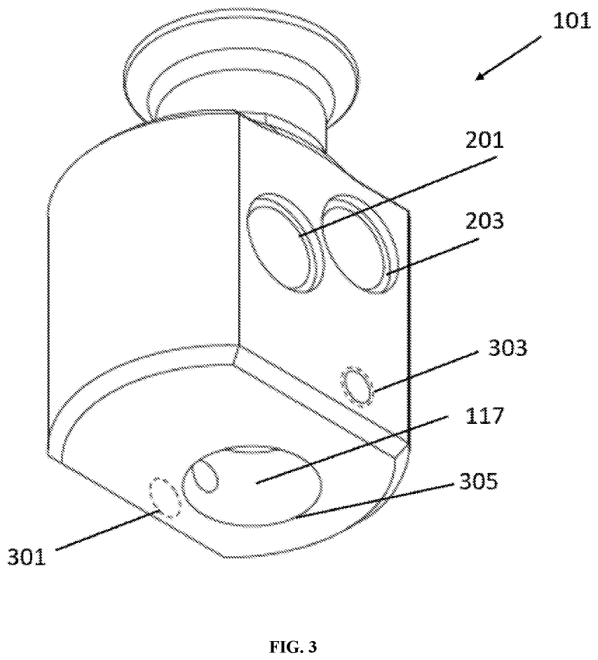

FIG. 3 is a bottom perspective view of member 105 of device 101. Member 105 may be configured to accept each of opposing screws 205, 207 through separate openings 301, 303. Coupling structure 117 may form an opening 305 for accepting the endoscope (not shown) so that device 101 can be mechanically coupled to the endoscope when in use. Coupling structure 117 and opening 305 may be further configured to allow endoscopy operating instrument 103 to pass from internal working channel 107 into the endoscope when in use.

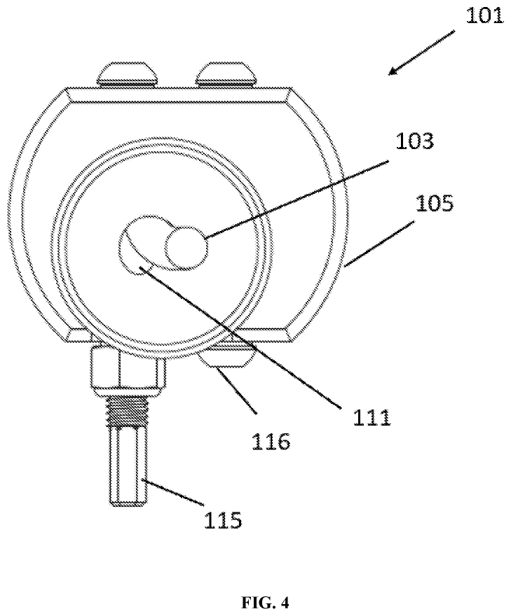

In FIG. 4, the orientation of endoscopy operating instrument 103 within internal working channel 107 is shown while device 101 is in use. The endoscopy operating instrument 103 is rotated as it is advanced through the device 101. Rotation is done by the helical shape of internal working channel 107 and by the off-set configuration of pins 115, 116. For example, the pair of pins 115, 116 are configured such that the endoscopy operating instrument is disposed between them when it is being advanced, and such that the pins each have an axis of rotation. At least one of the pins 115 is mechanically actuated to rotate. For advancing the endoscopy operating instrument 103 in a first direction towards the posterior end of the device 101, pin 115 is mechanically actuated to rotate clockwise. This clockwise rotation causes the endoscopy operating instrument 103 to advance towards the posterior end of the device 101, and also causes pin 116 to rotate counterclockwise. As the endoscopy operating instrument is advanced, it contacts the walls of the internal working channel 107. Since the internal working channel 107 has a helical shape, the endoscopy operating instrument 103 is rotated as it advances though the internal working channel 107 and repeatedly contacts the walls of the internal working channel 107.

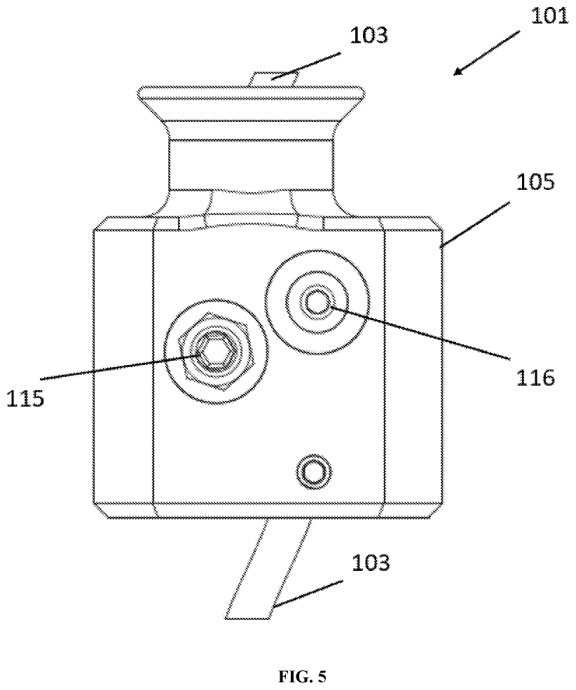

In FIG. 5, the orientation of the endoscopy operating instrument 103 within internal working channel 107 is shown. As discussed above, and as can be seen in FIG. 5, endoscopy operating instrument 103 may be rotated as it is propelled through the internal working channel 107. The rotation is mediated by the helical shape if internal working channel 107 and by off-set parallel pins 115, 116. As pins 115, 116 propel the endoscopy operating instrument 103 through internal working channel 107, endoscopy operating instrument 103 rotates.

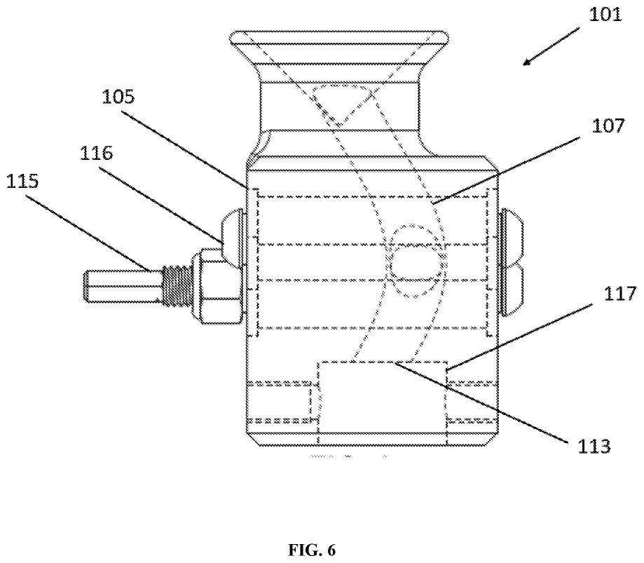

FIG. 6 shows the helical configuration of the internal working channel 107, the off-set configuration of the parallel pins 115, 116, the location of the exit port 113 with respect to the internal working channel 107 and coupling structure 117. FIG. 6 also shows the configuration of the coupling structure 117 configured to receive the endoscope, and the orientation of the openings 301, 303 for the screws 205, 207 used for coupling the device 101 to the endoscope.

Pins 115, 116 of device 101 of FIGS. 1-6 are configured in an offset orientation. As discussed above, rotation of endoscopy operating instrument 103 is mediated by the helical shape if internal working channel 107 and by the offset of pins 115, 116. As pins 115, 116 propel the endoscopy operating instrument 103 through internal working channel 107, endoscopy operating instrument 103 rotates. When in use, pin 115 may be configured to communicate with a controller (not shown). The controller can actuate parallel pin 115 to rotate in one of two directions and controls the rate of rotation and/or duration of rotation. Pin 116 is configured to passively rotate when pin 115 is actuated and as the endoscopy operating instrument passes between pins 115 and 116. Also, the distance between parallel pins 115 and 116 can be adjusted to a predetermined distance such that endoscopy operating instruments of varying sizes, shapes, dimensions and/or diameters can be accommodated by device 101.

FIG. 7 is a schematic showing device 101 coupled to an endoscope 701. Device 101 may be coupled to a channel opening structure 703 on endoscope 701. The endoscopy operating instrument 103 may be manually fed by an operator into device 101. Once endoscopy operating instrument 103 is inserted into device 101, pins 115, 116 may be actuated to propel endoscopy operating instrument 103 through device 101, and into channel opening structure 703 of the endoscope. From channel opening structure 703, endoscopy operating instrument 103 enters the insertion tube 705 of the endoscope. Endoscopy operating instrument 103 is propelled through insertion tube 705 and exits through a distal end 707 of insertion tube 705. Pin 115 is mechanically coupled to a controller 709 which is activated by the operator. Controller 709 actuates pin 115 so that endoscopy operating instrument 103 is mechanically propelled. Controller 709 is configured to control pin 115 such that it can instruct pin 115 to rotate in a first direction to propel endoscopy operating instrument 103 towards distal end 707 of the insertion tube 705, or in a second direction to pull the endoscopy operating instrument 103 away from the distal end 707 of the insertion tube 705 and back towards device 101. As discussed above, the combination of the off-set parallel pins 115, 116 and the helical configuration of internal working channel 107 allow for the rotation of the endoscopy operating instrument 103 as it is propelled from device 101 and through the insertion tube 705. Endoscopy operating instrument 103 continues to rotate once it exits through the distal end 707 of the insertion tube 705.

FIG. 7 shows a system according to an embodiment of the invention. In FIG. 7, the device 101 of FIG. 1 is coupled to an endoscope 701 and to a controller 709. The device 101 is coupled to a channel opening structure 703 on the endoscope 701. The endoscopy operating instrument is manually fed by an operator into the device 101. Once the endoscopy operating instrument 103 is inserted into the device 101, the pins 115, 116 are actuated to propel the endoscopy operating instrument 103 through the device 101, and into the channel opening structure 703 of the endoscope 701. From the channel opening structure 703, the endoscopy operating instrument 103 enters the insertion tube 705 of the endoscope 701. The endoscopy operating instrument 103 is propelled through the insertion tube 705 and exits through a distal end 707 of the insertion tube 705. Pin 115 is mechanically coupled to a controller 709 which is activated by the operator. The controller 709 actuates pin 115 so that the endoscopy operating instrument 103 is mechanically propelled.

The controller 709 is configured to control the pin 115 such that it can instruct pin 115 to rotate in a first direction to propel the endoscopy operating instrument 103 towards the distal end 707 of insertion tube 705, or in a second direction to pull the endoscopy operating instrument 103 away from the distal end 707 of insertion tube 705 and back towards the device 101. As discussed above, the combination of the off-set pins 115, 116 and the helical configuration of the internal working channel 107 allow for the rotation of the endoscopy operating instrument 103 as it is propelled from the device 101 and through the insertion tube 705. The endoscopy operating instrument 103 continues to rotate once it exits through the distal end 707 of the insertion tube 705.

The controller 709 actuates pin 115. The controller actuates pin 115 to rotate in one of two directions and controls the rate of rotation and/or duration of rotation of pin 115. By controlling the rate of rotation and/or duration of rotation of pin 115, the controller regulates the speed at which the endoscopy operating instrument 103 is passed through the endoscope 701 and/or the distance endoscopy operating instrument 103 is passed through the endoscope 701 and out from the distal end of the insertion tube 705. Pin 116 is configured to passively rotate when pin 115 is active as the endoscopy operating instrument 103 passes between pins 115 and 116.

The controller 709 can be programmed to actuate pin 115 under the settings of a predetermined protocol. By way of non-limiting example, the predetermined protocol can contain parameters regarding the rate of rotation of pin 115, the direction of rotation of pin 115, and/or the duration of the rotation of pin 115. Such parameters facilitate use of the endoscopy operating instrument 103 during surgical procedures and facilitate cleaning of the insertion tube 705 following a surgical procedure.

For instance, during a surgical procedure, the insertion tube 705 of the endoscope 701 can be inserted into a patient to within a predetermined distance from a target tissue. Then, the controller 709 can be actuated to execute a first predetermined program instructing the feeding system 114 of the device 101 to propel the endoscopy operating instrument 103 for a predetermined period of time such that the endoscopy operating instrument 103 is passed through the device 101, through the insertion tube 705, and to the target tissue for execution of the surgical procedure. Once the surgical procedure is completed, the controller 709 can be actuated to execute a second predetermined program which instructs the feeding system 114 to pull the endoscopy operating instrument 103 from the target tissue, back through the insertion tube 705, and out from the device 101. The first and second predetermined programs are adjusted to account for the type of endoscope used for the procedure. For example, the predetermined programs can be set to actuate the feeding system for a shorter duration of time to accommodate a shorter endoscope (e.g. a gastroscope). Alternatively, the predetermined programs can be set to actuate the feeding system for a longer duration of time to accommodate a longer endoscope (e.g. a colonoscope). In some embodiments, the predetermined programs allow for about 1 complete rotation of the endoscopy operating instrument per 100 cm.

Upon completion of the procedure, the endoscopy operating instrument 103 is removed from the device 101 and replaced with a cleaning cable (not shown) having a brush for cleaning the insertion tube 705. Once the cleaning cable is inserted into the device 101, the controller 709 can be actuated to execute a third predetermined program (i.e. a “cleaning program”) which allows the cleaning cable (not shown) to pass through the insertion tube 705 at an increased rate. This increased rate causes an increase in the frequency of rotation of the cleaning cable (not shown) as compared to the frequency of rotation of the endoscopy operating instrument 103 for efficient cleaning of the insertion tube 705. In some embodiments, the cleaning program is configured to allow for 1, 2, 3, 4, or 5 complete rotations of the cleaning cable 100 cm.

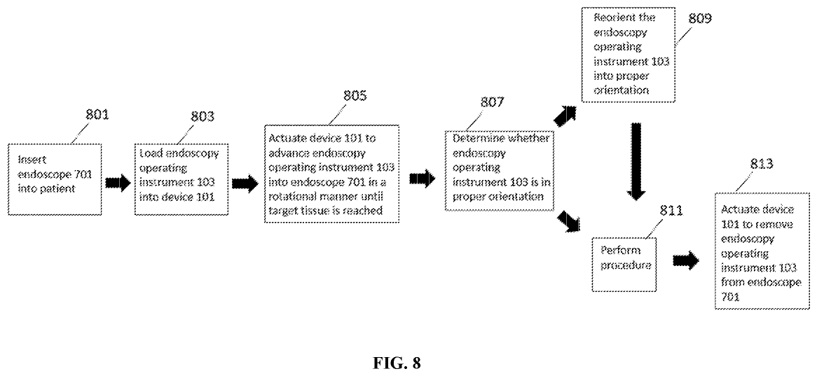

FIG. 8 is a flow chart depicting a method of use of the device and system described above for advancing an endoscopy operating instrument into an endoscope during an endoscopy according to an embodiment of the invention. First, the endoscope 701 is inserted into the patient 801. Once the target tissue is reached, the endoscopy operating instrument 103 manually loaded into the device 101, 803. Next, the feeding system 114 of the device 101 is actuated by an operator to advance the endoscopy operating instrument 103 through the internal working channel 107 of the device 101, and into the working channel (not shown) of the endoscope 701 in a rotational manner 805. The feeding system 114 of the device 101 is a pair of pins 115, 116. The pair of pins 115, 116 are configured such that the endoscopy operating instrument 103 is disposed between them when it is being advanced. At least one of the pins 115 is mechanically actuated to rotate. Upon actuation of the pin 115, the endoscopy operating instrument 103 is advanced through the internal working channel 107 of the device 101. The endoscopy operating instrument 103 is rotated as it is advanced through the device 101. Rotation is done by the helical shape of internal working channel 107 and by the configuration of the pins 115, 116, as discussed above.

The endoscopy operating instrument 103 is advanced through the insertion tube 705 of the endoscope 701 until it reaches the target tissue. Upon reaching the target tissue, the operator evaluates if the endoscopy operating instrument 103 is in the correct orientation for the procedure 807. If the endoscopy operating instrument 103 is not in the correct orientation for the procedure, the operator adjusts the endoscope 701—for example, by moving it away from the target tissue—and actuates the device 101 to once again feed the endoscopy operating instrument 103 in a rotational manner such that the endoscopy operating instrument 103 is reoriented in the correct orientation 809. Once the endoscopy operating instrument 103 is in the correct orientation, the procedure is carried out 811.

Upon completion of the procedure, the operator actuates the device to remove the endoscopy operating instrument 103 from the target tissue by pulling the endoscopy operating instrument 103 through the insertion tube 705, out of the endoscope 701, and out of the device 101, 813.

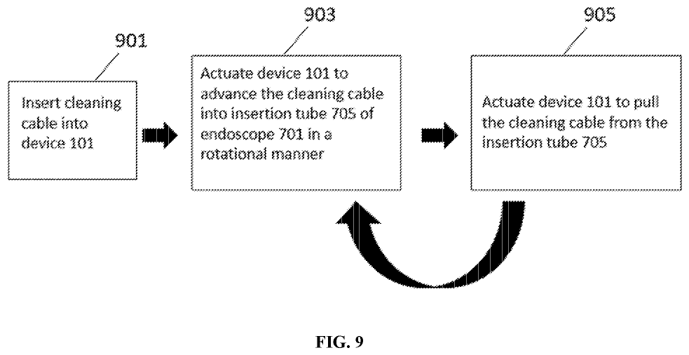

FIG. 9 is a flow chart depicting a method of use of the device and system described above for cleaning an endoscope according to an embodiment of the invention. First, a cleaning cable (not shown) having a brush for cleaning the insertion tube 705 is manually inserted into the device 101, 901. Once the cleaning cable is inserted into the device 101, an operator actuates the feeding system 114 of the device 101 so that the endoscopy operating instrument 103 is advanced through the internal working channel 107 of the device 101, and into insertion tube 705 in a rotational manner, 903. The feeding system 114 can be actuated to propel the cleaning cable (not shown) to pass through the insertion tube 705 at an increased rate. This increased rate causes an increase in the frequency of rotation of the cleaning cable (not shown) as compared to the frequency of rotation of the endoscopy operating instrument 103 for efficient cleaning of the insertion tube 705. The feeding system can also be actuated to pull the cleaning cable from the insertion tube 705, 905. This process is repeated for a predetermined number of intervals, or until the insertion tube 705 is sufficiently cleaned.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

We claim:

1. A device for advancing a medical instrument through an endoscope, comprising:
a housing configured to be coupled to a proximal end of said endoscope; and
a mechanism in said housing configured to advance said medical instrument in a rotational manner through said housing and into said endoscope.

2. The device of claim 1, wherein said housing forms a working channel passing through said housing, and wherein said working channel is configured to extend between an inlet and an outlet.

3. The device of claim 2, wherein said mechanism in said housing is configured to advance the medical instrument in a rotational manner comprises a pair of rotatable pins coupled to said housing and configured to propel the medical instrument through said working channel.

4. The device of claim 3, wherein said pair of rotatable pins are offset.

5. The device of claim 2, wherein said working channel comprises a helical shape.

6. A device for feeding an endoscopy operating instrument into an endoscope, comprising:
a member forming an internal working channel, said internal working channel configured to surround a portion of said endoscopy operating instrument, and said member further comprising:
an input structure positioned at an anterior end of said member;
an entry port formed by said input structure, said entry port providing access to a first end of said internal working channel;
an exit port disposed on a posterior end of said member, said exit port providing access to a second end of said internal working channel;
a feeding system mechanically coupled to said member, said feeding system configured to propel said endoscopy operating instrument through said internal working channel; and
a coupling structure positioned at a posterior end of said member, said coupling structure configured to mechanically couple said member to said endoscope.
wherein said internal working channel comprises a helical shape, and
wherein said feeding system is further configured to propel said endoscopy operating instrument in a first direction from said anterior end of said member to said posterior end of said member, and alternately in a second direction from said posterior end of said member to said anterior end of said member.

7. The device of claim 6, wherein said internal working channel is configured to rotate said endoscopy operating instrument between 1 and 10 rotation(s) per 100 centimeters as said feeding system propels said endoscopy operating instrument through said internal working channel.

8. The device of claim 6, wherein said feeding system is configured to be in communication with a controller.

9. The device of claim 6, wherein said feeding system is a pair of parallel pins.

10. The device of claim 9, wherein said pair of parallel pins are offset from each other.

11. The device of claim 9, wherein said pair of parallel pins comprise a drive wheel, and an idle wheel.

12. The device of claim 9, wherein said pair of parallel pins are configured such that said endoscopy operating instrument passes between said pair of parallel pins when said device is in use.

13. The device of claim 9, wherein at least one of said pair of parallel pins is configured such that said at least one of said pair of parallel pins can be adjusted to be a predetermined distance from a second of said pair of parallel pins.

14. The device of claim 6, wherein said input structure comprises a funnel shape.

15. A system for feeding an endoscopy operating instrument into an endoscope, comprising:
a controller; and
a device for feeding said endoscopy operating instrument, said device comprising:
a member forming and internal working channel, said internal working channel configured to surround a portion of said endoscopy operating instrument, and said member further comprising:
an input structure positioned at an anterior end of said member;
an entry port formed by said input structure, said entry port providing access to a first end of said internal working channel;
an exit port disposed on a posterior end of said member, said exit port providing access to a second end of said internal working channel;
a feeding system mechanically coupled to said member, said feeding system configured to propel said endoscopy operating instrument through said internal working channel; and
a coupling structure positioned at a posterior end of said member, said coupling structure configured to mechanically couple said member to said endoscope,
wherein said internal working channel comprises a helical shape,
wherein said feeding system is configured to be in communication with a controller, and
wherein said feeding system is further configured to propel said endoscopy operating instrument in a first direction from said anterior end of said member to said posterior end of said member, and alternately in a second direction from said posterior end of said member to said anterior end of said member.

16. The system of claim 15, wherein said controller is configured instruct said feeding system to propel said endoscopy operating instrument for at least one of a predetermined length of time, a predetermined distance, and a predetermined speed.

17. The system of claim 15, wherein said internal working channel is configured to rotate said endoscopy operating instrument between 1 and 10 rotation(s) per 100 centimeters as said feeding system propels said endoscopy operating instrument through said internal working channel.

18. The system of claim 15, wherein said feeding system is a pair of parallel pins.

19. The system of claim 18, wherein said pair of parallel pins are offset from each other.

20. The system of claim 18, wherein said pair of parallel pins comprise a drive wheel, and an idle wheel.

21. The system of claim 18, wherein said pair of parallel pins are configured such that said endoscopy operating instrument passes between said pair of parallel pins when said device is in use.

22. The system of claim 18, wherein at least one of said pair of parallel pins is configured such that said at least one of said pair of parallel pins can be adjusted to be a predetermined distance from a second of said pair of parallel pins.

23. The system of claim 15, wherein said input structure comprises a funnel shape.

24. A method of feeding an endoscopy operating instrument into an endoscope comprising:
coupling the device of claim 1 to said endoscope;
feeding said endoscopy operating instrument into the device of claim 1; and
actuating the device of claim 1 such that said device of claim 1 advances said endoscopy operating instrument into said endoscope in a rotational manner.

25. A method of cleaning an endoscope, comprising:
coupling the device of claim 1 to an endoscope;
feeding a cleaning instrument into the device of claim 1;
actuating the device of claim 1 such that said device of claim 1 advances said cleaning instrument into said endoscope in a rotational manner; and
actuating the device of claim 1 such that said device of claim 1 pulls said cleaning instrument from said endoscope.
